(19) United States
(12) Patent Application Publication
(10) Pub. No.: US 2026/0191693 A1
(43) Pub. Date: Jul. 9, 2026

(54) BANDAGE WITH METAL LAYER

(71) Applicant: LCP Medical Technologies, LLC, Rogers, MN (US)

(72) Inventor: James Rathburn, Rogers, MN (US)

(21) Appl. No.: 19/441,734

(22) Filed: Jan. 6, 2026

Related U.S. Application Data
(60) Provisional application No. 63/742,349, filed on Jan. 6, 2025.

(51) Int. Cl. A61F 13/02; A61F 13/0203

(52) U.S. Cl. CPC A61F 13/0289; A61F 13/022

(57) ABSTRACT

Embodiments for a method for creating a bandage are provided. A sheet of absorbent material having a first face and a second face and defining a plurality of pores exposed at the first face for absorbing liquid from a wound is provided. Particle-free metal ink is deposited on the first face of the sheet of absorbent material. The particle-free metal ink is cured on the first face of the sheet of absorbent material to form metal bonded to the sheet of absorbent material at the first face.

### RELATED APPLICATIONS

This application claims the benefit of U.S. Provisional Application No. 63/742,349, filed on Jan. 6, 2025, entitled “WOUND CARE USING PARTICLE-FREE METAL AND/OR METAL-OXIDE INK”, the contents of which are hereby incorporated herein by reference.

### BACKGROUND

Traditional wound care is predominantly comprised of two types of bandages or wound dressings. One is an adhesive-bearing fabric or polymer film with a non-woven polymer absorbent pad covered by a non-adhering perforated contact layer to allow for fluid to pass and reduce the potential for adhesion to the wound as it heals. A second type is a wound dressing, which is a sheet of absorbent material that is held in place against the wound with a binding material such as tape or some other wrapping that acts as retention against the wound. In some cases, antiseptic or antimicrobial ointments are added to aid with moisture retention and provide some healing enhancement and bacterial content reduction to avoid infection.

In some cases, wound care treatments are enhanced by adding silver or silver derivatives to the contact medium to increase the dressing's antiseptic/antimicrobial effect. These treatments can be enhanced with ointments that contain silver or silver-bearing materials or, in some cases, zinc oxide particles. It is a commonly held principle that free ion exchange from these metal or metal oxide-bearing materials disrupts the cell walls of infectious bacteria and pathogens, eliminating the ability to replicate with a reduction in infection risk.

In some cases, metal or metal oxide materials are contained within the dressing material, such as spun-bonded or melt-blown polymers or fabrics. One type is called Silverlon, and it is comprised of a sheet of nylon fabric with silver-plated fibers. Several commercial sources sell a variety of dressings containing silver nitrate, silver sulfadiazine, or other silver-related compositions. These dressings are passive and rely on the release of ions over time that encounter the fluids exuded from wounds and any additional ointments.

Vomaris is a company that produces one case that takes a more active approach. It leverages the galvanic reaction principles of dissimilar metals located in proximity to each other and in contact with an activation medium, such as the fluid at the wound site. Silver particles are placed into the fiber matrix of the wound dressing, with Zinc particles placed in close proximity to the silver particles. When subjected to fluids or saline, for example, the metals exchange free ions in a galvanic reaction that creates a minor current and voltage and is essentially a self-initiating battery effect. This reaction creates an environment that also disrupts pathogen cell walls to eliminate reproduction as well as creates the electrical effect that promotes healing and attraction-movement of regenerative cells.

### BRIEF DESCRIPTION

In some embodiments, a method for creating a bandage is provided. A sheet of absorbent material having a first face and a second face and defining a plurality of pores exposed at the first face for absorbing liquid from a wound is provided and particle-free metal ink is deposited on the first face of the sheet of absorbent material. The particle-free metal ink is cured on the first face of the sheet of absorbent material to form metal bonded to the sheet of absorbent material at the first face.

In other embodiments, a bandage is provided. The bandage includes a sheet of absorbent material having a first face and a second face and defining a plurality of pores exposed at the first face for absorbing liquid from a wound. The bandage also includes a layer of metal bonded to the first face of the sheet of absorbent material, wherein the layer of metal maintains openings to the plurality of pores at the first face, such that the sheet of absorbent material can absorb liquid from a wound through the first face.

### BRIEF DESCRIPTION OF THE DRAWINGS

Understanding that the drawings depict only exemplary embodiments and are not therefore to be considered limiting in scope, the exemplary embodiments will be described with additional specificity and detail through the use of the accompanying drawings, in which:

FIGS. 1A and 1B are cut-away cross-sectional views of example stages in the creation of an adhesive bandage according to one inventive embodiment;

FIGS. 2A and 2B are top and bottom views of the example adhesive bandage 100 of FIGS. 1A and 1B;

FIGS. 3A and 3B are cut-away cross-sectional views of example stages in the creation of a non-adhesive bandage according to an inventive embodiment;

FIG. 4 is a cut-away cross-sectional view of an example stage in the creation of another adhesive bandage according to an inventive embodiment;

FIG. 5 is a cut-away view of a cross-sectional view of an example stage in the creation of yet another adhesive bandage according to an inventive embodiment; and

FIGS. 6-8 are top views of example patterns of first and second particle-free metal inks on the adhesive material of the adhesive bandage or the base material of bandages of FIGS. 1-5.

### DETAILED DESCRIPTION

A significant limitation of existing silver-based wound dressings is the cost of silver and the expense of manufacturing. The use of silver or a silver derivative alone does have an impact on infection risk reduction while lacking significant electrical properties to enhance wound healing. It is believed that the silver plus zinc configuration, which creates a mild voltage or electric field, is more effective in infection reduction and nerve and tissue regeneration. This existing bandage has limitations as the particles are bound to the dressing matrix and can have an adhesive or binder barrier that insulates the particle and reduces conductivity and ion exchange when in contact with fluids. These particles can also dislodge and enter the wound environment. Another limitation of existing bandages is the relative distance between the zinc and silver deposits. This can create ineffective regions where fluids must bridge the physical space to initiate the ion exchange and electrical effect and reduce the benefits. There is a hydrogel application to these existing bandages required to initiate any electrical - biometric healing effects.

The subject matter described herein focuses on the benefits of adding metal-metal oxide to a conventional passive wound care dressing or bandage while leveraging the simplicity and high-volume usage of commercial bandages. In particular, the subject matter described herein applies a unique and novel approach to wound care by utilizing the same basic components and material sets used in conventional bandage applications and uses particle-free metal inks to add metal to the bandages. The particle-free metal inks can, when converted, provide pathogen elimination conditions as well as galvanic ion exchange to enhance wound healing and tissue regrowth.

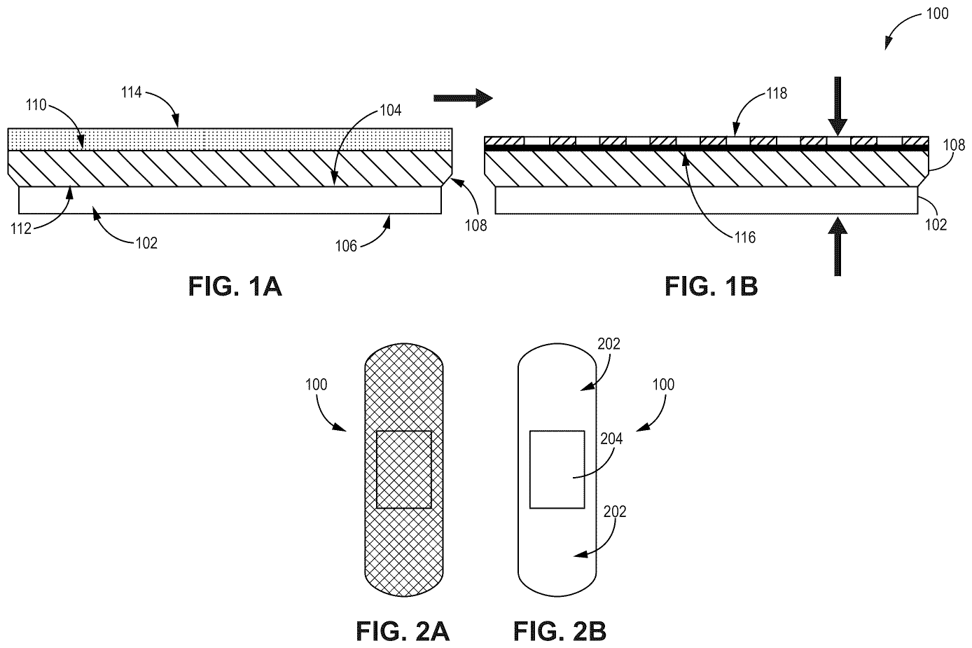

FIGS. 1A and 1B are cut-away cross-sectional views of example stages in the creation of an adhesive bandage 100 according to one inventive embodiment. In FIG. 1A, a sheet of base material 102 is provided, onto which adhesive and an absorbent pad can be disposed. The base material 102 is a flexible sheet of material that can conform to a human body for application thereto. Any base material suitable for use as the base of an adhesive bandage can be used, such as a fabric or polymer film. The sheet of base material 102 defines a first face 104 and a second face 106.

A sheet of absorbent material 108 is bonded to the base material 102 to form an absorbent pad on the base material 102. The sheet of absorbent material 108 defines a first face 110 and a second face 112. The sheet of absorbent material 108 is disposed on the base material 102 such that the base material 102 extends outward from the sheet of absorbent material 108 on at least one side thereof. FIGS. 2A and 2B are top and bottom views of an example adhesive bandage 100 showing portions 202 of the base material 102 that extend outward from the absorbent pad 204. The sheet of base material 102 can be disposed on the sheet of absorbent material in the same manner as existing adhesive bandages where the base material 102 extends outward on at least two opposing sides, and optionally on all sides of the sheet of absorbent material 108. The adhesive bandage 100 can have other shapes and sizes than that shown in FIGS. 2A and 2B.

The sheet of absorbent material 108 can be bonded to the base material 102 in any suitable way as known to those skilled in the art. A second face 112 of the sheet of absorbent material 108 can be bonded to the first face of the sheet of base material 102. Any absorbent material suitable for use on an adhesive bandage for absorbing liquids from a wound can be used, such as a non-woven polymer. The absorbent material is porous in that it defines a plurality of pores that open to the first face 110 thereof for absorbing liquids from a wound.

Adhesive (not shown) can be applied to one or more portions of first face 104 of the sheet of base material 102. In particular, the adhesive can be applied to one or more portions of the base material 102 that extend outward from the absorbent pad 204. The adhesive can be used to adhere the bandage to human skin. Any adhesive suitable for use on an adhesive bandage for adhering the adhesive bandage to skin and any manner of applying the adhesive on the base material 102 can be used.

Referring back to FIG. 1A, particle-free metal-bearing ink 114 can be disposed on the first face 110 of the sheet of absorbent material 108. Particle-free metal ink is a liquid that includes metal ions, metal molecules, and/or metal salts along with volatile solvents in a solution. The metal ions, molecules, and/or salts in the particle-free metal ink have a size less than 1 micron. In an example, the particle-free metal ink 114 can be disposed onto the sheet of absorbent material 108 by printing (e.g., with an inkjet printer), spraying, contacting the first face 110 with an ink pad, or any other appropriate method of dispensing the ink 114. The particle-free metal ink 114 can be comprised of Silver, a silver bearing composition, Zinc, and Zinc bearing composition, Copper, a Copper bearing composition or a combination of these metal bearing types. In a preferred embodiment zinc-based particle-free metal ink is used due to its lower cost compared with silver and copper.

The particle-free metal ink 114 can be cured via a thermal process to cause the metal therein to deposit in a layer 116 on the first face 110 of the absorbent material 108 as shown in FIG. 1B. There may be some dispersion within the fiber matrix of the absorbent material 108. A plasma atmosphere can be used during the curing to reduce oxidation potential, although some oxide formation is acceptable.

In an example, saline solution can be disposed on the first face 110 of the sheet of absorbent material 108 after curing the metal ink 114 thereon. The saline solution can be dried to form a saline-based deposit on the first face 110. The saline deposit can enable a reactive state and add reactivity when encountering wound fluid. The saline application is to be thin and limited to quantity and content that does not irritate the wound. Any suitable saline solution can be used.

A perforated contact layer 118 can be disposed over the sheet of absorbent material 108, including over the metal layer 116 and saline deposit (if present) thereon. Any performed contact layer 118 suitable for use on an absorbent pad of an adhesive bandage can be used. The contact layer 118 allows for air passage, fluid passage, and exposure of the metalization on the absorbent material 108 to wound fluids while reducing the risk of metal migration into the wound and limiting adhesion to the wound tissue during the healing process.

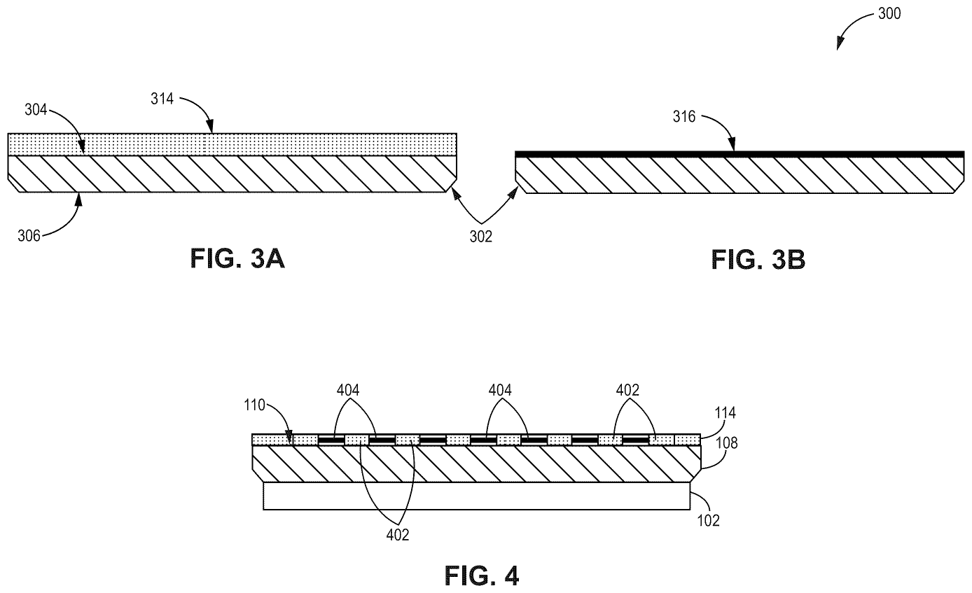

FIGS. 3A and 3B are cut-away cross-sectional views of example stages in the creation of a non-adhesive bandage 300. The non-adhesive bandage 300 includes a sheet of base material 302 similar to that described with respect to bandage 100. The base material 302 is a flexible sheet of material that can conform to a human body for application thereto. Any base material suitable for use as the base of a non-adhesive bandage can be used, such as a fabric, liquid crystal polymer (LCP) film, or polyolefin film. The sheet of base material 302 defines a first face 304 and a second face 306.

A particle-free metal-bearing ink 314 can be disposed on the first face 304 of the sheet of base material 302. In an example, the particle-free metal ink 314 can be disposed onto the sheet of base material 302 by printing (e.g., with an inkjet printer), spraying, contacting the first face 304 with an ink pad, or any other appropriate method of dispensing the ink 314. The particle-free metal ink 314 can be comprised of Silver, a silver bearing composition, Zinc, and Zinc bearing composition, Copper, a Copper bearing composition or a combination of these metal bearing types. In a preferred embodiment zinc-based particle-free metal ink is used due to its lower cost compared with silver and copper.

The particle-free metal ink 314 can be cured via a thermal process to cause the metal therein to deposit in a layer 316 on the first face of the base material 302 as shown in FIG. 3B. There may be some dispersion within a fiber matrix of the base material 302. A plasma atmosphere can be used during the curing to reduce oxidation potential, although some oxide formation is acceptable. This can form a layer of metal on the surface of the base material 302, as well as partial penetration of the surface or full saturation of the base material 302.

In an example, saline solution can be disposed on the first face 304 of the sheet of base material 302 after curing the metal ink 314 thereon. The saline solution can be dried to form a saline-based deposit on the first face 304. The saline deposit can enable a reactive state and add reactivity when encountering wound fluid. The saline application is to be thin and limited to quantity and content that does not irritate the wound. Any suitable saline solution can be used.

The non-adhesive bandage 300 can be held over a wound via external binding such as tape or wrapping. This non-adhesive bandage 300 can be configured for small wounds or large area wounds such as burns and bed sores. This non-adhesive bandage 300 can be wrapped around the wound area and held in place with conventional methods such as tape or non-adhesive wrapping.

FIG. 4 is a cut-away cross-sectional view of another example stage 400 in the creation of an adhesive bandage. The example stage 400 is the same as the stage shown in FIG. 1A, except the layer of ink 114 includes multiple different particle-free metal inks disposed on the first face 110 of the adhesive material 108. In some embodiments of the examples shown in FIGS. 1-3, a single particle-free metal ink is disposed on the base or adhesive material. In the example shown in FIG. 4, however, multiple different particle-free metal inks 402, 404 are interspersed amongst each other on the base or adhesive material. In these examples, a first particle-free metal ink 402 having at least a first metal therein is disposed one or more first portions of the first face of the base or adhesive material and a second particle-free metal ink 404 having at least a second metal therein is disposed in one or more second portions of the first face of the base or adhesive material. The first particle-free metal ink 402 does not include the second metal and the second particle-free metal ink 404 does not include the first metal. In this way, multiple dissimilar metals can be disposed on the base or adhesive material in proximity to each other to create a galvanic, free ion exchange environment to encourage, stimulate, and enhance nerve and tissue growth while healing. The first and second particle-free metal inks 402, 404 can disposed by spraying to provide precision material content control or printed (e.g., inkjet) across the surface or in specific patterns rather than the global coverage of spray coating. Specific pattern inkjet printing has the advantages of reduced material usage and localized arrangement of material sets to achieve galvanic reactions with dissimilar metals located in close proximity. In one embodient, the first particle-free metal ink 402 includes silver and the second particle-free metal ink 404 includes zinc. In another embodiment, the first particle-free metal ink 402 includes copper and the second particle-free metal ink 402 includes zinc. Selective application and position control reduce the risk of metal migration into the wound environment while generating the self-initiating battery effect to promote healing and eliminate infectious pathogens. The metal-based particle-free inks can be selectively printed onto the absorbent pad member as one embodiment, and the materials can be selectively printed on mating layers such as copper printed onto the absorbent pad and zinc printed onto the underside of the perforated contact layer such that when mated the metal patterns are in specific proximity to each other with limited direct contact to the wound surface yet in contact with wound fluids. Although two different particle-free metal inks are shown, more than two different particle-free metal inks could be deposited on the first face 110.

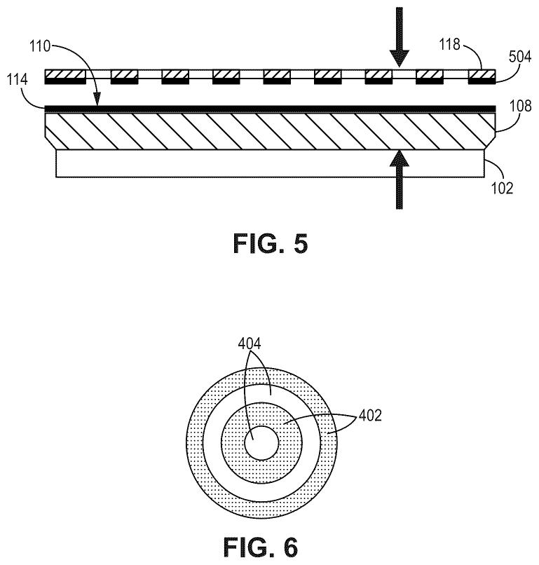

FIG. 5 is a cut-away cross-sectional view of another example stage 500 in the creation of an adhesive bandage. The example stage 500 is the same as the stage shown in FIG. 1B, except there is a first particle-free metal ink 114 disposed on the first face 110 of the adhesive material 108 and a second particle-free metal ink 504 disposed on the perforated contact layer 118. In this examples, a first particle-free metal ink 114 having at least a first metal therein is disposed one or more first portions of the first face of the base or adhesive material and a second particle-free metal ink 504 having at least a second metal therein is disposed in one or more second portions of the first face of the base or adhesive material. The first particle-free metal ink 114 does not include the second metal and the second particle-free metal ink 504 does not include the first metal. In this way, multiple dissimilar metals can be disposed on the base or adhesive material in proximity to each other to create a galvanic, free ion exchange environment to encourage, stimulate, and enhance nerve and tissue growth while healing. The first and second particle-free metal inks 114, 504 can disposed by spraying to provide precision material content control or printed (e.g., inkjet) across the surface or in specific patterns rather than the global coverage of spray coating. Specific pattern inkjet printing has the advantages of reduced material usage and localized arrangement of material sets to achieve galvanic reactions with dissimilar metals located in close proximity. In one embodient, the first particle-free metal ink 114 includes silver and the second particle-free metal ink 504 includes zinc. In another embodiment, the first particle-free metal ink 502 includes copper and the second particle-free metal ink 114 includes zinc. Selective application and position control reduce the risk of metal migration into the wound environment while generating the self-initiating battery effect to promote healing and eliminate infectious pathogens. The metal-based particle-free inks can be selectively printed onto the absorbent pad member as one embodiment, and the materials can be selectively printed on mating layers such as copper printed onto the absorbent pad and zinc printed onto the underside of the perforated contact layer such that when mated the metal patterns are in specific proximity to each other with limited direct contact to the wound surface yet in contact with wound fluids.

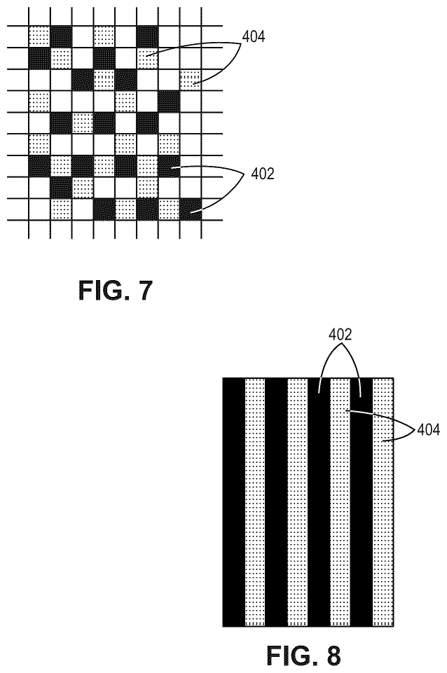

FIGS. 6-8 are top views of example patterns of particle-free metal ink disposed on a face of the base or adhesive material in any of the examples show in in FIGS. 1-5. FIGS. 6-8, however, show example patterns where multiple different particle-free metal inks are interspersed amongst each other on the base or adhesive material similar to that shown in cross-section in FIG. 4. Other patterns than that shown in FIGS. 6-8 can be used.

The subject matter herein has many benefits. From simple cuts to chronic wounds, diabetic legions, surgical incisions, and wounds at large, the invention's ability to eliminate infectious pathogens, enhance wound healing, and promote nerve and tissue growth with the use of the galvanic reaction of metal-based particle-free inks is significant. The commercial applications are similar to existing wound care, with a manufacturing process that leverages particle-free metal base dink formulations and proven printing and converting processes.

The use of Zinc and/or copper-based particle-free inks has significant cost and manufacturing benefits over silver-based methods with anticipated improvement in the promotion of wound healing.

The printing process is significantly less expensive than silver plating and has the advantage of selective deposition of various metal or metal oxide inks in proximity to each other to initiate free ion exchange.

The particle-free metal-bearing ink, when converted, is a conformal coating that binds to the support media, for example, the absorbent pad, and has greatly reduced the risk of particle migration into the wound site.

The manufacturing process can utilize existing high volume, low cost materials and packaging already used in the wound care and bandage market.

The use of Zinc and/or Zinc oxide based inks leverages the existing antiseptic ointment method of reducing the risk of wound infection and promotion of healing.

The ability to print various metal-based inks in proximity to each other in very thin and controlled environments has significantly effective bio-electric properties over particle-based methods that require a hydrogel to initiate the reaction.

A very thin, non-irritating saline coating can be applied to the printed metal-based particle-free ink deposition to hold the reaction in place and enhance the bioelectric function once in contact with wound fluids without requiring hydrogel, although hydrogel usage is also possible with the subject matter herein.

What is claimed is:

1. A method for creating a bandage, the method comprising:
providing a sheet of absorbent material having a first face and a second face and defining a plurality of pores exposed at the first face for absorbing liquid from a wound;
disposing particle-free metal ink on the first face of the sheet of absorbent material; and
curing the particle-free metal ink on the first face of the sheet of absorbent material to form metal bonded to the sheet of absorbent material at the first face.

2. The method of claim 1, comprising:
providing a sheet of base material having a first face and a second face;
bonding the second face of the sheet of absorbent material to the first face of the sheet of base material, the sheet of base material extending outward from the sheet of absorbent material such that sheet of absorbent material forms an absorbent pad on the sheet of base material and defines at least a first portion of the sheet of base material that extends outward from the absorbent pad; and
disposing adhesive on a portion of at least a first portion of the first face of the sheet of base material, wherein the adhesive is configured to adhere the bandage to human skin.

3. The method of claim 2, comprising:
disposing a perforated contact layer over the first face of the sheet of absorbent material including over the metal bonded to the sheet of absorbent material, such that the perforated contact layer is disposed between the sheet of absorbent material and the wound when the bandage is applied to human skin.

4. The method of claim 1, wherein disposing particle-free metal ink on the first face of the sheet of absorbent material includes one or more of printing, spraying, and contacting with an ink pad.

5. The method of claim 1, wherein the particle-free metal ink includes one or more of silver, zinc, and copper.

6. The method of claim 1, comprising:
providing a plasma atmosphere proximate the particle-free ink during the curing.

7. The method of claim 1, comprising:
disposing a saline solution on the first face of the sheet of absorbent material after curing the particle-free metal ink on the first face; and
drying the saline solution on the first face of the sheet of absorbent material to form a saline deposit at the first face of the sheet of absorbent material.

8. The method of claim 1, wherein disposing particle-free metal ink on the first face of the sheet of absorbent material includes:
disposing a first particle-free metal ink on one or more first portions of the first face of the sheet of absorbent material; and
disposing a second particle-free metal ink on one or more second portions of the first face of the sheet of absorbent material,
wherein the second particle-free metal ink includes at least one metal that is not in the first particle-free metal ink and the first particle-free metal ink includes at least one metal that is not in the second particle-free metal ink.

9. The method of claim 8, wherein the one or more first portions of the first face of the sheet of absorbent material are interspersed amongst the one or more second portions of the first face of the sheet of absorbent material.

10. The method of claim 3, comprising:
wherein disposing particle-free metal ink on the first face of the sheet of absorbent material includes depositing a first particle-free metal ink on the first face of the sheet of absorbent material;
disposing a second particle-free metal ink on a first face of the perforated contact layer;
curing the second particle-free metal ink on the first face of the perforated contact layer, wherein disposing the perforated contact layer over the first face of the sheet of absorbent material includes disposing the first face of the second particle-free metal ink facing the first face of the sheet of absorbent material,
wherein the second particle-free metal ink includes at least one metal that is not in the first particle-free metal ink and the first particle-free metal ink includes at least one metal that is not in the second particle-free metal ink.

11. A bandage comprising:
a sheet of absorbent material having a first face and a second face and defining a plurality of pores exposed at the first face for absorbing liquid from a wound; and
a layer of metal bonded to the first face of the sheet of absorbent material, wherein the layer of metal maintains openings to the plurality of pores at the first face, such that the sheet of absorbent material can absorb liquid from a wound through the first face.

12. The bandage of claim 11, comprising:
a sheet of base material bonded to the second face of the sheet of absorbent material, the sheet of base material extending outward from the sheet of absorbent material such that sheet of absorbent material forms an absorbent pad on the sheet of base material and defines at least a first portion of the sheet of base material that extends outward from the absorbent pad;
adhesive disposed on a portion of at least a first portion of the first face of the sheet of base material, wherein the adhesive is configured to adhere the bandage to human skin.

13. The bandage of claim 12, comprising:
a perforated contact layer disposed over the first face of the sheet of absorbent material including over the metal bonded to the sheet of absorbent material, such that the perforated contact layer is disposed between the sheet of absorbent material and the wound when the bandage is applied to human skin.

14. The bandage of claim 11, wherein the metal includes one or more of silver, zinc, and copper.

15. The bandage of claim 11, comprising:
a saline deposit at the first face of the sheet of absorbent material.

16. The bandage of claim 11, wherein the layer of metal bonded to the first face of the sheet of absorbent material includes:
one or more first portions including a first metal and one or more second portions including a second metal, wherein the one or more first portions do not include the second metal and the one or more second portions do not include the first metal.

17. The bandage of claim 16, wherein the one or more first portions are interspersed amongst the one or more second portions.

18. The bandage of claim 13, comprising:
a layer of metal on a first face of the perforated contact layer, wherein the first face of the perforated contact layer faces the first face of the sheet of absorbent material, wherein the layer of metal bonded to the first face of the sheet of absorbent material includes a first metal and not a second metal, wherein the layer of metal on the first face of the perforated contact layer includes a second metal and not a first metal.
